(19) United States
(12) Patent Application Publication
(10) Pub. No.: US 2014/0336475 A1
(43) Pub. Date: Nov. 13, 2014

(54) Methods and Means for Clinical Investigations

(71) Applicant: JPWaVe, Amersfoort (NL)

(72) Inventor: Johan Renes, Amersfoort (NL)

(21) Appl. No.: 14/309,748

(22) Filed: Jun. 19, 2014

Related U.S. Application Data
(63) Continuation of application No. 12/927,521, filed on Nov. 16, 2010, now abandoned.

(51) Int. Cl. A61B 5/00

(52) U.S. Cl. CPC A61B 5/4848; A61B 5/0022; A61B 5/6898; A61B 5/7282
USPC 600/301; 600/300

(57) ABSTRACT

Described are methods for testing at least one effect of a pharmaceutical substance in a subject, which include administering a pharmaceutical substance to the subject, measuring with at least one sensor contained within a mobile sensor system, at the subject or in close proximity to the subject, at least one parameter value indicative of a the subject's body function, transmitting at least one sensor system signal associated with the at least one parameter value to a receiver contained within a mobile base unit, the receiver being provided with a means for wireless transmission, and wirelessly transmitting a mobile base unit signal associated with the at least one sensor system signal from the receiver to a back-end system, wherein at least the back-end system correlates the at least one parameter value with and/or displays a representation of the at least one effect of the pharmaceutical substance.

### CROSS-REFERENCE TO RELATED APPLICATION(S)

This application is a continuation of co-pending U.S. patent application Ser. No. 12/927,521, filed Nov. 16, 2010, pending, the disclosure of which is hereby incorporated herein by this reference in its entirety.

### TECHNICAL FIELD

The disclosure relates to the field of medicine generally. In particular, it relates to the field of clinical trials (or clinical experiments utilizing live subjects) for the development of substances and/or devices useful for diagnosing, preventing, ameliorating and/or treating undesired (pathological) conditions. It combines knowledge from two distant fields: (1) the field of clinical development and (2) the field of mobile data capture and data transmission technology. An important feature is this combination of these two distant fields. As described herein, the inventor has realized that the combination of these fields provides inter alia more accurate and/or more reliable data for testing or clinical testing of a pharmaceutical substance and/or medical device(s). Provided is the application of mobile data capture and data transmission technology in the field of clinical development or drug development.

### BACKGROUND

Numerous instances exist where circumstances can dictate, or at least influence, outcome. Easy calculus that one can answer from the top of one's head may present an insurmountable problem under stress conditions (e.g., as shown in exams and television game shows). The reverse is also true. Tasks that seem insurmountable under “normal” conditions, can be accomplished if the need is extremely high to achieve them (e.g., during life-threatening situations). Undoubtedly, a strong interaction exists between body and mind. This is also true for our health. “Mens sana in corpore sanum.” Herpes simplex clinical episodes (e.g., the occurrence of cold sores) are strongly correlated with stress. In medicine, the so-called “placebo-effect” is clear evidence of the strength of the mind in influencing outcome of a treatment.

Thus, it seems clear that the mind plays an important role in the efficacy of drugs. Yet, when drugs are tested for their clinical efficacy, the mind is not just typically ignored as a factor, it is in fact set in an artificial, typically stressful environment. When drugs are tested in human subjects (e.g., as part of clinical development), such tests are typically carried out in a “hospital” setting, which, for most people, is a setting associated with negative emotions. Where these “hospital” conditions may not significantly affect the efficacy of, e.g., antibiotics, one may expect that there are numerous classes of drugs of which the efficacy is influenced by the state of mind of the user. Examples are psychotropic drugs, hormones, and drugs for metabolic diseases, such as insulin.

### DISCLOSURE

Provided are methods and means for testing at least one effect of at least one pharmaceutical substance in at least one subject, the methods comprising measuring with at least one measuring device at the subject or in close proximity to the subject, at least one parameter indicative of a body function of the at least one subject, transmitting the measurement to at least one receiving device, the receiving device being provided with a means for wireless transmission, and transmitting the measurement from the receiving device to a computer (back-end system), wherein at least the computer correlates the at least one parameter with and/or displays a representation of the at least one effect of the at least one pharmaceutical substance.

Further provided is a monitoring system for testing an effect of a pharmaceutical substance in a subject, the system comprising a mobile body area network and a remote user network, wherein the mobile body area network comprises a sensor system and a mobile base unit and the remote user network comprises a back-end system, wherein the sensor system is suitable for determining a parameter value of a subject, the parameter value being associated with the effect of the pharmaceutical substance, and for transmitting a sensor signal to the mobile base unit, the sensor signal being associated with the parameter value, and the mobile base unit is suitable for receiving the sensor signal and for wirelessly transmitting a mobile base unit signal associated with the sensor signal to the back-end system, wherein the back-end system comprises a service controller, an on-line service center and an interface to a digital vault, the service controller being arranged for automatically preparing an exact and secured copy of the at least one mobile base unit signal associated with the received sensor signal and transmitting the copy to the digital vault, and the on-line service center being arranged for correlating the at least one sensor signal with at least one effect of the pharmaceutical substance.

Also disclosed is a method for mobile monitoring, the method comprising: detecting at least one parameter value and wirelessly transmitting a sensor signal associated with the at least one parameter value using at least one mobile sensor system carried by a movable object or movable organism; receiving the sensor signal from the at least one mobile sensor system and wirelessly transmitting a mobile base unit signal associated with the received sensor signal using a mobile base unit carried by the movable object or movable organism; receiving the mobile base unit signal using a back-end system, thus establishing a wireless communications link between the mobile base unit and the back-end system; and making data associated with the mobile base unit signal available to a user.

### BRIEF DESCRIPTION OF THE DRAWINGS

FIG. 1 shows the set-up of a monitoring system for use in a clinical test setting, wherein data are stored without the necessity of human interference.

FIG. 2 shows an overview of a study design. The administration of medication X and placebo is described in a fixed order, but in reality, administration is randomized.

### DETAILED DESCRIPTION

As used herein, a “pharmaceutical substance” is any substance having an effect on a subject that is considered useful in the amelioration, prevention and/or treatment of an undesired (pathological) condition of the subject.

As used herein, a “substance” is defined as a chemical compound, such as an inorganic or organic chemical compound including, but not limited to, peptides, proteins, steroids, polysaccharides, nucleotides, combinations of the aforementioned compounds or compositions comprising the aforementioned compounds or combinations thereof.

As used herein, a “subject” is defined as a human or animal or a model representing a human or animal or a part of a human or animal. A subject thus is to be understood as a subject to be monitored, not necessarily having a disorder. It will be appreciated that it is also possible to monitor healthy people, for instance, healthy people who require special attention, such as sportsmen, elderly or infants.

As used herein, a “measuring device” is defined as a means arranged to determine at least one value of a parameter of the subject through the translation of physical and/or chemical parameters into an electromagnetic signal.

Parameters indicative of a body function may be any parameters that can be measured through a sensor that may be used at or near a subject. It is also possible to use parameters that are measured inside a subject's body, although non-invasive sensors may be used. The parameters that can be measured include, but are not limited to, heart rate, blood pressure, blood flow, blood oxygenation, body temperature, glucose levels, levels of other chemicals and/or biologicals, conductivity (e.g., of skin), etc. Typically, a sensor will measure a parameter and convert it into an electrical current, which can be relayed and/or converted into digital data. Conversion into digital data may be accomplished by methods known in the art. The meaning and scope of the term “digital data” is considered to be well known in the art. The important feature here is that the signal, which correlates to an effect on a parameter measured at or near the subject, is converted into transferable, storable and interpretable digital data that still correlate to the same effect. A sensor may be reusable or disposable. A sensor may be connected to a measuring device or it may be an integral part of a measuring device. The measuring device may communicate (wireless) with a receiving device or it may be an integral part of a receiving device.

As used herein, multiple (at least two) parameters may be measured at or near the same subject, which may be measured simultaneously. The different sensors needed for these measurements may be connected to one or more measuring/receiving devices. The measurements for one subject may be relayed through a single receiving device. Measurements may be made continuously or at certain intervals. Signals from the measurements may be relayed immediately or they may be stored in a storage device before being relayed. The storage device may be separate or integrated into one of the other devices. Signals may also be analyzed by the measuring device. When such an analysis is made, measurements may be relayed upon a certain event in the measurements, or for a certain interval of time surrounding a certain event in the measurements. In this way, a large bulk of signals and/or data containing less relevant data (or less immediately relevant data) need not be relayed, or may be relayed at a later moment in time. As an example, when measuring heart rate, it may not be necessary to relay measurements as long as the heart rate remains within a certain range. Once it is outside the range, signals/data of the heart rate sensor and/or measuring device may be relayed to the receiving device or if the analysis takes place at the receiving device, from the receiving device onward. The signals and/or data relayed at that time may be originating from the sensor making the heart rate measurement, but also from other sensors/devices measuring parameters at or near the same subject. It may be relevant to determine what caused the event (in this case change in heart rate), therefore, the measuring device and/or receiving device may retrieve some of its stored historical data/signals and relay these onward in the system together with or preceding/following the data at the time of the event that triggered the relay. Sensors and/or measuring devices may also be provided that measure parameters from the environment of the subject. For instance, the temperature of the surroundings of the subject may be relevant for certain parameters measured from the subject.

As used herein, a “receiving device” is defined as a means arranged for receiving (directly or indirectly) a signal from at least one measuring device and for wirelessly transmitting a signal correlated with the signal received from the measuring device. The receiving device may be a separate or integrated device. It may be integrated with the measuring device (which in itself may be integrated with the sensor(s)) and/or it may be integrated (or part of) a storage/computing/analyzing device. It may be connected (hard-wired or wireless) to a separate storage/computing/analyzing device. It may be a pda or a mobile phone. It may have a user interface. It may be connected to, or have a monitor and/or a printing device. The important part is that in a system, as used herein, there may be provided for a user interface at or near the site where the sensors are measuring. This may be a unidirectional, or two-way or multidirectional interface. This interface connects the subject or a person near the subject (user) with a person (responsible) at the back-end of the system (directly or indirectly). The responsible person may be able to direct, question and/or instruct the user (subject). The user (and/or subject) may be able to ask questions or send messages to the responsible person.

As used herein, the term “computer” has its usual meaning, including a system comprising, e.g., several connected CPUs and/or several connected storage facilities. Typically, a computer includes a display and input devices such as a keyboard and a mouse.

A sensor may measure chemical and/or physical parameters. To be useful, these measurements have to be converted into electric (electromagnetic) signals and/or digital data. The electric signals may be filtered, amplified and/or modified in any manner considered necessary and/or useful. The signals may be converted into digital data at any stage of the processes described herein. This may be done in the measuring device, the receiving device, or the back-end computer. The digital data can be further processed (filtered, analyzed) also at any stage. They may be partially stored and partially relayed, completely stored for later transmission, etc.

The methods and means disclosed herein inter alia provide the following advantages in the field of clinical development. Pharmaceutical substances (hereinafter also referred to as drugs) under clinical development can now be tested under conditions resembling their eventual “circumstance of use” more closely, instead of restricting the clinical testing of drugs to hospital-like environments wherein subjects are limited in their behavior. This limit may bias the outcome of the clinical tests. For example, the clinical efficacy of statins is known to be dependent on behavioral components and/or environmental influences, such as the amount of physical exertion, stress, and food intake. In a hospital-like setting, these components and or influences will typically deviate from routine behavior.

As used herein, data can be collected anywhere and anytime, thereby allowing for any type of circumstances (behavioral and/or environmental) considered useful and/or relevant for the analysis of a drug/device under clinical testing. Such circumstances, optionally including behavioral and/or dietary instructions, include, but are not limited to, a home-setting, a less-restricted hospitalized-setting (e.g., more freedom of movement in and around the hospital), free-roaming (e.g., essentially unrestricted movement), testing under exertion, the natural and/or regular habitat of the subject, or combinations thereof, thereby allowing the support of the development of more “personalized medicine.”

“Personalized medicine” is based upon the emerging knowledge that the effect of medicines as measured on the basis of large groups of subjects (by statistical analysis) may be different than the effects on smaller groups or individual subjects. In large groups treated as one group, there may actually be two subgroups, one of which has no real benefit of a drug and a second group that has a strong benefit of the drug. By treating these two groups as one, the effects of the drug are underestimated for the group in need of the drug (if the group is smaller, it may even be insignificant) or overestimated for the other group. Improved subgrouping of patients allows for more tailored drug treatment and better solutions. The availability of tailor-designed clinical studies will support the improved clinical development of new and also existing drugs to the right patient at the right time.

Provided herein are means and methods suitable to be combined with other (innovative) systems that are used for screening for the presence of subgroups within a larger group. Since all data may be made available without human interference (and interpretation), such analyses become more feasible and more reliable.

As used herein, integrity of data can be increased. Collected data may be relayed immediately to a (central) data storage facility without the possibility of any human interference. Such a data storage facility may be at or under control of the end-user (e.g., a pharmaceutical company) and/or at or under control of an escrow-like body and/or at or under control of a regulatory body such as the FDA or EMEA.

Paper-free information may be instantly and constantly compiled as part of a real-time growing database. Clinical trial subjects can be continuously monitored by a remote clinical research center while continuing normal mobility under normal life circumstances. Drug efficacy and safety profiles can be tested in an individualized home-like setting enabling the collection of physical and mental measures under conditions more closely representing conditions under which drugs are actually being used. Individualized dialogues with clinical subjects is possible and in real-time. A strong commitment to completing post-marketing studies can be accomplished. An increased public commitment to drug safety and surveillance can be expected. The corporate governance of drug developing companies is supported. Clinical trial subject compliance and persistency clinical trial is supported. The public's perception of the value of medicines becomes more apparent.

Although the instant disclosure explains in more detail applications relating to human clinical trials, the same disclosure also applies to veterinary medicine and the field trials therein, and to animal experiments preceding or supporting human clinical trials. As explained hereinbefore, the disclosure is not limited to pharmaceuticals per se, but can also be applied for testing diagnostic devices, medical devices, and other devices for which behavior of the subject interacting with it may be relevant; especially objects and/or devices related to well being are good candidates for testing. For example, recently, a new kind of alarm clock was introduced by Philips, which wakes a person up in what is stated to be a more pleasant way. The instant disclosure could show whether this is actually the case by measuring relevant parameters of subjects waking up under their normal “home” conditions.

In situations where animals are subjected to tests (either as target subjects or as pre-stage for human testing) it may be very well feasible to use transmission systems that are wireless, but function only within a short distance or within in a confined space (for instance, provided with signaling triggers, such as wiring in floors, walls and/or ceilings). In certain circumstances, such a set-up may also be useful for human trials. The disclosure enables trials in subjects (both human and other) under circumstances that can be chosen by the investigator. Depending on the risks associated with the test, the subjects involved, the need to have contact with the subjects, etc., a test set-up may be chosen ranging from a confined situation to a completely free-roaming subject test and everything in between. There are, of course, certain constraints associated with every different set-up. In a free-roaming set-up, a subject may be out of reach of mobile networks for a certain period of time. This more or less excludes high-risk tests to be carried out under such conditions. It also means that storage capacity is needed at or near the subject. It also means that certain measurements will need to be assigned to a certain moment in time or a certain event. This calls in the case that different measurements are made for a means for synchronization of measurement. Provided is a means to achieve synchronization without actually having to synchronize.

As used herein, the use of a mobile monitoring system is provided, the system comprising at least one mobile sensor system arranged for detecting a parameter value and wirelessly transmitting a sensor signal associated with the parameter value, a mobile base unit arranged for receiving the sensor signal from the at least one mobile sensor system and for wirelessly transmitting a mobile base unit signal associated with the received sensor signal, wherein the at least one mobile sensor system and the mobile base unit are arranged to be carried by a movable object or movable organism, wherein the mobile monitoring system further comprises a back-end system arranged for receiving the mobile base unit signal, thus allowing a wireless communications link between the mobile base unit and the back-end system, and making data associated with the mobile base unit signal available to a user.

It will be appreciated that herein a signal, such as the sensor signal, may have data associated therewith, e.g., data associated with the parameter value, and may be in digital form.

Thus, the mobile base unit can be wirelessly connected to one or a plurality of mobile sensor systems. This provides the advantage that the mobile sensor system or systems can be easily applied to the movable object or movable organism, e.g., subject, while the mobile base unit can separately be applied to the subject, e.g., worn on the clothing or carried in a pocket of a person's clothing, without the need of any physical connection between the mobile sensor system or systems and the mobile base unit. Further, a user, such as a physician, may monitor the data associated with the mobile base unit signal, which may comprise data relating to the detected parameter value, via the wireless communications link and via the back-end system.

It is possible to communicate data from the mobile base unit towards the back-end system and vice versa. It is, for instance, possible for the physician using the back-end system, e.g., via a user terminal and an interface, such as a web-application, to transmit commands, such as feedback, e.g., tactile, audio, and/or visual feedback signals, to the subject carrying the mobile base unit.

In certain embodiments, the back-end system comprises a service controller arranged for automatically preparing an exact and secured copy of the mobile base unit signal associated with the at least one received sensor signal and transmitting the copy to a digital vault. The digital vault may only be accessible to regulatory authorities, like FDA or EMEA. The service controller may further transmit the mobile base unit signal associated with the at least one received sensor signal to an on-line service center for correlating the at least one sensor signal with at least one effect of the pharmaceutical substance. The on-line service controller may prepare a representation of the at least one sensor signal with the at least one effect of the pharmaceutical substance automatically and/or on-demand for visualization on an external data display.

The mobile base unit may be further arranged for wirelessly transmitting a mobile-base-unit-to-sensor-system signal, e.g., associated with the received back-end system signal, and the at least one mobile sensor system is arranged for receiving the mobile-base-unit-to-sensor-system signal. It is possible to activate the actuator by receiving a command from the mobile base unit or from the back-end system via the mobile base unit. Thus, it is, for instance, possible for the physician using the back-end system, e.g., via a user terminal and an interface, such as a web-application, to transmit commands, such as feedback, e.g., tactile, audio, and/or visual feedback signals, to the sensor system via the mobile base unit. Also, it is possible for the subject using the mobile base unit to transmit commands to the sensor system.

In certain embodiments, the mobile base unit is arranged such that it can be wirelessly connected to different mobile sensor systems, e.g., determining values of different parameters. Thus, a very versatile system is provided.

The mobile base unit may comprise a first plug-in software module for converting the first sensor data into the first converted sensor data and/or a second plug-in software module for converting the second sensor data into the second converted sensor data. Thus, it is possible to allow any type of sensor signal to be converted into a properly formatted converted sensor signal, which may be uniform for all converted sensor signals, by providing the correct plug-in software module.

The mobile base unit may be arranged for gathering data associated with received sensor signals into a data record, and for processing the data record. Here, processing may, e.g., comprise transmitting of the data record. Thus, a well-defined data structure may be provided.

The mobile base unit may be arranged for gathering the sensor data associated with received sensor signals into a data record.

The mobile base unit may be arranged, during consecutive predetermined storage intervals, for each predetermined storage interval accumulating the sensor signal or all data associated with that sensor signal received during that predetermined storage interval, and transmitting the mobile base unit signal associated with the accumulated sensor signal or data associated with the received sensor signal after lapse of that storage interval. A plurality of data records may be accumulated during that predetermined storage interval. This provides the advantage that data may be transmitted from the mobile base unit to the back-end system in batches, which may, e.g., reduce power consumption of the mobile base unit.

In a generally applicable embodiment, the mobile base unit comprises an indicator for indicating data associated with the received sensor signal and/or data associated with the received back-end system signal to the user and/or movable organism. The indicator may comprise a display, and the data associated with the received sensor signal and/or associated with the received back-end system signal is indicated to the user and/or movable organism via a user interface.

The data associated with the received sensor signal and/or the data associated with the received back-end system signal may be communicated to the user interface using the Internet Protocol (IP).

In certain embodiments, the mobile base unit may comprise a non-volatile memory for storing the data associated with the received sensor signal. Thus, in addition to transmitting data associated with the received sensor signals to the back-end system, such data may also be stored into the memory. All data associated with all received sensor signals may be stored into the memory. This provides the possibility of retrieving any data, e.g., in case of loss of data due to malfunction or transmission errors in the wireless communication to the back-end system.

The mobile base unit may be arranged to transmit historical data stored in the memory of the mobile base unit, e.g., data not previously transmitted, upon request by the back-end system or when triggered by a special event. Thus, it is possible to selectively omit transmission of data associated with certain, or certain parts of, received sensor signals and transmit previously non-transmitted data when needed. This may, e.g., be useful in case of a medical episode, in which a user of the back-end system is interested in data predating the onset of the medical episode by a certain amount of time.

The at least one mobile sensor system may be connected or connectable to the mobile base unit via a short-range wireless communications connection, such as BLUETOOTH®. It will be appreciated that if a plurality of mobile sensor systems is communicatively connected to the mobile base unit, different wireless communication connections, e.g., of different types, may be used side by side.

In certain embodiments, at least one of the mobile sensor systems comprises an event button and is arranged for transmitting a sensor signal associated with a status of the event button.

Upon receiving the mobile base unit data record, the back-end system interprets the data record and may search for event-related data. If this data is available, the back-end system may create an event message (e.g., text, audio, and/or visual) and may forward this message to a third party service provider: text to SMS or email service provider, audio/visual to MMS or phone service provider, or Internet application provider.

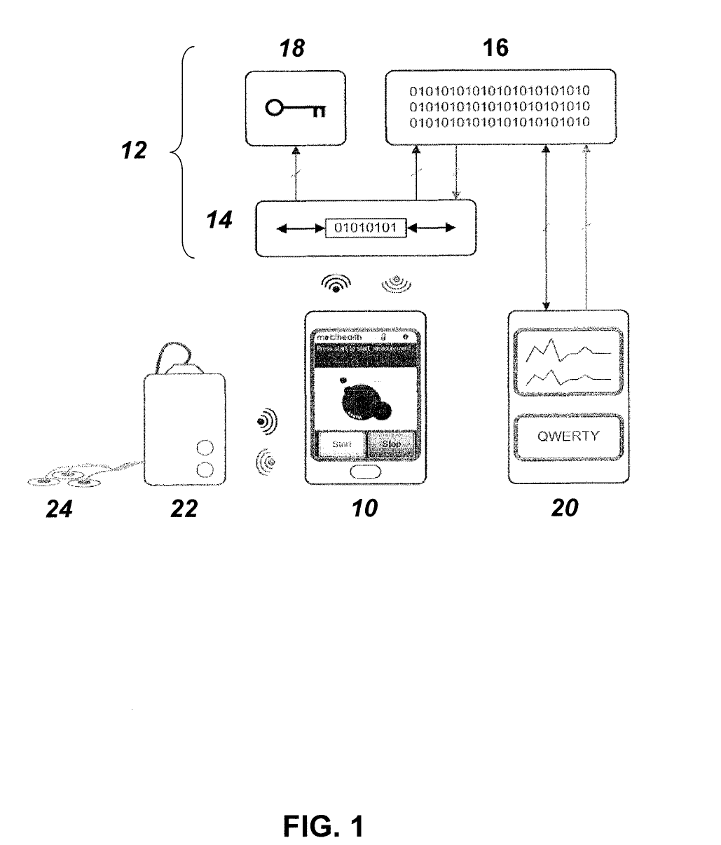

According to the embodiment of FIG. 1, the mobile base unit wirelessly transmits all physiological data to the back-end system. The back-end system as shown in FIG. 1 comprises a service controller and an on-line service center. The service controller automatically prepares an exact and secured copy of the original physiological data, and transmits this copy to a digital vault. In this way, human intervention does not occur, and data tampering is prevented. The digital vault may only be accessible to regulatory authorities, like FDA or EMEA. In addition, the service controller transmits the original physiological data to the on-line service center. In the on-line service center, the data may be (automatically) processed by the authority responsible for conducting the pharmaceutical investigation, for instance, into a suitable format for analysis, e.g., correlation of physiological data with the effect of a pharmaceutical substance and presentation. The data may be displayed, e.g., on-line, on a data display device, for instance, a personal computer with visualization software. From the data display device, a signal for controlling the process of the measurement of physiological data, e.g., controlling measurement interval, start time, stop time, may transmitted to the mobile base unit without the original physiological data from the sensor system being processed.

The invention is further described with the aid of the following Examples.

### Example 1

### Efficacy of Medicine X on the Subjective and Vaginal Sexual Response to Erotic Stimuli in Women with Hypoactive Sexual Desire Disorder

In a double-blind, randomly assigned placebo controlled cross-over design, a group of 32 women with hypoactive sexual desire disorder (“HSDD”) are tested with Medicine X and of placebo.

### Medication

Medication X=on demand medication for HSDD with maximum efficacy one to three hours after intake.

Placebo=a composition having the same shape, color, odor, taste, route of administration, etc., as Medicine X, but without the active ingredient.

### Measurements

1. Vaginal pulse amplitude (“VPA”) and subjective rating of erotic stimuli are measured in response to erotic film clips in the institutional laboratory, under condition of Medicine X and Placebo.

2. VPA and subjective rating of erotic stimuli are measured in response to erotic film clips in the homes of the subjects with a mobile laboratory, under condition of Medicine X and Placebo.

3. Sexual functioning in general (e.g., regarding experiences of sexual encounters with spouse) are measured by a diary and a monthly questionnaire (e.g., Female Sexual Functioning Questionnaire; FSFI).

### Screening

The experiments are preceded by a screening visit. In this screening visit, subjects are interviewed and examined by a gynecologist to diagnose for FSD and to determine eligibility for study participation. Subjects are asked to fill out a questionnaire; the Female Sexual Function Index (“FSFI”). Subjects are screened to exclude pregnancy or breast feeding, vaginal infections, major operations to the vagina and/or vulva, undetected major gynecological illnesses or unexplained gynecological complaints. Weight, height, and blood pressure (supine and standing) are measured. Cardiovascular conditions are tested and ECG checked for significant abnormalities. Subjects are screened for a history of endocrinological, neurological or psychiatric illness and/or treatment. Standard blood chemistry and hematology tests are performed. Participants are required not to use alcohol or psychoactive drugs the evening before and the day of experimentation. During period of menstruation, subjects will not be tested.

### Laboratory Measurement (Institutional Lab and Mobile Lab)

The VPA is measured in response to neutral and erotic film excerpts, one hour after drug administration. The two experimental days are separated by (at least) a three-day period. On the two experimental days, subjects receive one capsule consisting of either Medicine X, or Placebo.

During the experimental sessions in the laboratory, subjects take Medication X or placebo, and after one hour, the subject must insert a tampon-shaped vaginal probe (a photoplethysmograph) in order to measure the VPA. Then subjects will view a ten-minute neutral fragment, followed by a five-minute erotic film fragment. Blood pressure (supine and standing), heart rate, respiration rate, and body temperature are monitored throughout on the experimental days.

### Measurements and Drug Administration at Home (Sexual Function Diary and Questionnaires)

One hour before a sexual experience at home (coitus, masturbation, etc.), medication is taken. After each sexual encounter, subjects write down their experiences in a diary. In this diary, the amount of arousal, desire, pain, orgasm intensity, etc., is described. Each month, subjects fill out the FSFI. This questionnaire measures experienced arousal, desire, pain, orgasm intensity, etc., over the past month.

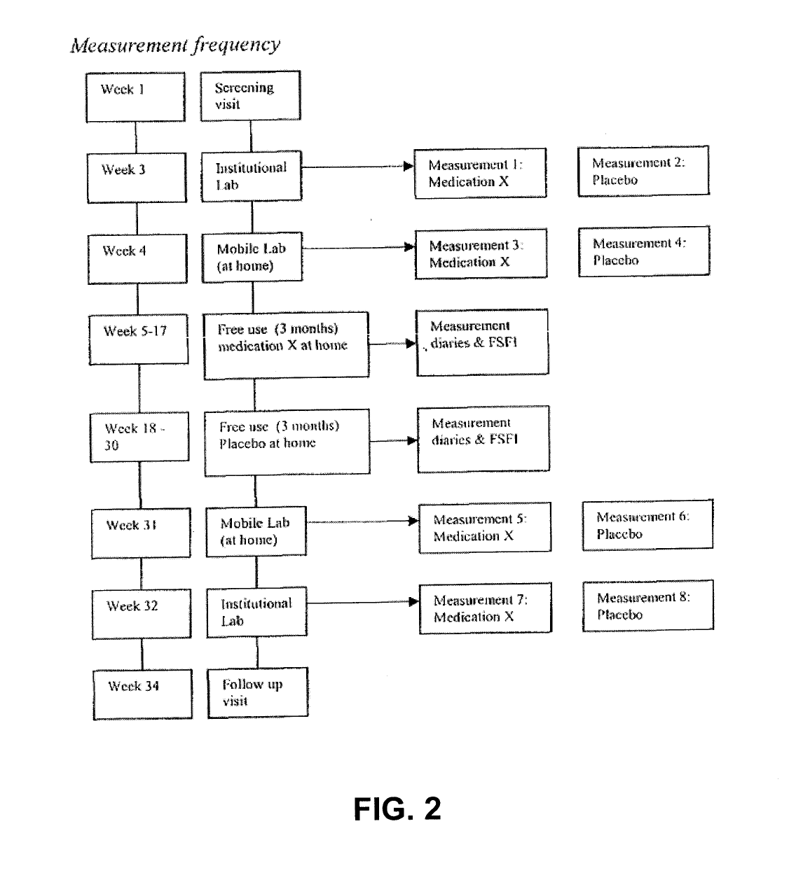

VPA and subjective sexual arousal to erotic film clips is measured four times in the institutional laboratory and four times at home (see FIG. 2). After the first two institutional measurements and the first two mobile lab measurements at home, subjects receive three months' medication X or placebo to take home for use in sexual encounters. After the first three months, subjects take home three months of medication of the second type. Then, two mobile lab measurements at home and two institutional measurements follow.

### Example 2

### Efficacy and Safety of Medicine Y Versus Placebo in Patients with ICD Implants

In a Randomized Parallel Group Trial, four groups of 24 patients each with an implantable cardioverter defibrillator (ICD) are tested under treatment of Medicine Y (groups 1 and 3) or Placebo (groups 2 and 4).

To assess in patients with CAD (coronary artery disease) and an ICD, the effect of Medicine Y (group 1) versus placebo on the first recurrence of a ventricular arrhythmia (ventricular tachycardia or ventricular fibrillation) requiring ICD within one year after randomization.

Groups 1 and 2: measurements in an institutional setting during three months after which they are measured at home during two to three months*.

Groups 3 and 4: measurements in home setting during three months after which they are measured in an institutional setting during two to three months*.

*Group sizes and the duration of the experiment may be amended for achieving statistical significance.

### Medication

Medication Y: 1 dd or 2 dd 100 mg medication for the Prevention of Ventricular Arrhythmia.

Placebo=a compound with the same shape, color, odor, taste, route of administration, etc., as Medicine Y, but without the active ingredient.

### Measurements

Primary: Time to all ventricular tachycardia or ventricular fibrillation arrhythmia leading to any ICD intervention (anti-tachycardia pacing or ICD shock). Institutional and home.

Secondary: Time to all arrhythmia episodes leading to at least one documented ICD shock. Institutional and home.

### Inclusion Criteria:

Patient with an ICD implanted during the previous year for documented spontaneous life-threatening ventricular arrhythmia OR implanted with an ICD and with at least one appropriate ICD therapy (shock or anti-tachycardia pacing) for ventricular tachycardia or ventricular fibrillation in the previous year.

Left ventricular ejection fraction measured by 2D-echocardiography must have been documented to be less than 40% in the last six months.

Have a relatively stable home situation.

### Exclusion Criteria:

MAIN CRITERIA (non-exhaustive list): see below.

### Screening

The experiments are preceded by extensive physical examination, ECG, and echocardiography. In this screening, subjects are interviewed and examined by a cardiologist to determine eligibility for study participation. Subjects are screened to exclude women of childbearing potential without adequate birth control, pregnant women, breastfeeding women, conditions that increase the risk of severe anti-arrhythmic drug side effects, and severe associated conditions. Weight, height, and blood pressure (supine and standing) are measured. Standard blood chemistry and hematology tests are performed. Participants may not use alcohol or psychoactive drugs.

### Laboratory Measurement (Institutional Lab and Mobile Lab)

The ECG is measured on a continuous (24/7) basis (for example, using the systems described in U.S. Pat. No. 7,197,357 and US2007/0078324, the contents of which are incorporated herein by this reference). Blood pressure (supine and standing), heart rate, respiration rate, and body temperature are monitored throughout on the experimental days.

### Drug Administration

1 dd or 2 dd 100 mg Medicine Y is administered per os. 2 dd administration entails equal distribution of the total doses over the day. Subjects are institutionalized for a period of two weeks after which they are measured at home for two weeks.

### Example 3

### Efficacy and Safety of Medicine Z Versus Placebo in the Treatment of Psychotic Symptoms in Patients with Major Depressive Disorder with Psychotic Features

Approximately 200 patients are distributed over four groups randomized to receive Medicine Z or placebo for seven days followed by antidepressant. The purpose is to compare the efficacy of Medicine Z administered in a home situation and in an institutional setting in reducing psychotic symptoms in patients with a diagnosis of psychotic depression.

Groups 1 and 2: measurements in an institutional setting during seven days after which they are measured at home during seven days*.

Groups 3 and 4: measurements in a home setting during seven days after which they are measured in an institutional setting during seven days*.

*Group sizes and the duration of the experiment may be amended for achieving statistical significance.

### Medication

Medication Z: 1 dd or 2 dd of an established dose of medication for the treatment of psychotic symptoms in patients with major depressive disorder with psychotic features.

Placebo=a compound with the same shape, color, odor, taste, route of administration, etc., as Medicine Y, but without the active ingredient.

### Measurements

Primary: The proportion of Medication Z versus placebo-treated patients who achieve a score reduction from baseline on a system for the measurement of sympathetic nervous system activity (institutional and home).

Secondary: The proportion of Medication Z-treated patients with plasma drug concentrations above a specified amount versus placebo-treated patients who achieve a score reduction from baseline on a system for the measurement of sympathetic nervous system activity (institutional and home).

### Inclusion Criteria:

Have a DSM-IV TR diagnosis of Major Depressive Disorder with Psychotic Features.

Are clinically symptomatic with their illness.

Have pre-specified minimum scores on standardized psychiatric rating scales at baseline.

Have not been taking excluded medication for at least seven days prior to randomization.

Have a relatively stable home situation (e.g., not in divorce or social separation, no recent bereavement).

### Exclusion Criteria:

Have any primary psychiatric diagnosis other than psychotic depression.

Have a major medical problem, which, in the opinion of the Investigator, would place the patient at undue risk.

Have undergone electroconvulsive therapy within three months prior to randomization.

Have had a hospitalization due to a suicide attempt within 45 days prior to randomization.

Are female and of childbearing age, and are unable or unwilling to use two medically acceptable methods of contraception during the study and for three months after study completion, one of which must be a barrier method.

Are female and are pregnant or lactating.

Are currently taking excluded medications.

Have used drugs of abuse within 30 days prior to screen, as per patient report and urine drug screen.

Have a history of active drug or alcohol abuse within three months or dependence within six months prior to screening.

Are in the opinion of the Investigator at immediate risk of suicide, or at risk of harming others.

Have received investigational therapy (drug, vaccine, biological agent or device) within six months prior to randomization.

Have previously participated in a clinical trial of Medicine Z.

Have a history of an allergic reaction to Medicine Z.

Are in the Investigator's opinion not appropriate for participation in the study or may not be capable of following the study schedule for any reason.

Are patients who are employees of the study unit or their family members, students who are working in the study unit, or family members of the Investigator.

### Screening:

The experiments are preceded by extensive physical and psychiatric examination and ECG. In this screening, subjects are interviewed and examined by a psychiatrist to determine eligibility for study participation. Weight, height, and blood pressure (supine and standing) are measured. Standard blood chemistry and hematology tests are performed. Participants are required not to use alcohol.

### Laboratory Measurement (Institutional Lab & Mobile Lab):

Parameters of sympathetic nervous system activity are measured on a continuous basis (for example, using the systems described in US2008/0165017, and/or Kinhy et al.; Schizophrenia Bulletin, published May 8, 2009; and/or Poh et al.; IEEE Transactions on Biomedical Engineering; 57; 5, 2010, the contents of which are incorporated herein by this reference). Blood pressure (supine and standing), heart rate, respiration rate, and body temperature are monitored throughout on the experimental days.

### Drug Administration

1 dd or 2 dd of an established dose of Medicine Z is administered. An established dose is considered to be the dose that is effective at acceptable safety level (side effects). 2 dd administration entails equal distribution of the total doses over the day. Subjects are institutionalized for a period of seven days after which they are measured at home for seven days.

1. A method for testing at least one effect of a pharmaceutical substance in a subject, the method comprising:
administering a pharmaceutical substance to the subject,
measuring with at least one sensor contained within a mobile sensor system, at the subject or in close proximity to the subject, at least one parameter value indicative of a body function of the subject,
transmitting at least one sensor system signal associated with the at least one parameter value to a receiver contained within a mobile base unit, the receiver being provided with a means for wireless transmission, and
wirelessly transmitting a mobile base unit signal associated with the at least one sensor system signal from the receiver to a back-end system, wherein at least the back-end system correlates the at least one parameter value with and/or displays a representation of the at least one effect of the pharmaceutical substance.

2. The method according to claim 1, further comprising:
transmitting a back-end system signal utilizing the back-end system and receiving the back-end system signal utilizing the mobile base unit.

3. The method according to claim 2, further comprising:
wirelessly transmitting a mobile base unit-to-sensor-system signal, and
receiving the mobile-base-unit-to-sensor-system signal utilizing the at least one mobile sensor system.

4. The method according to claim 3, comprising:
detecting a first parameter value and a second parameter value,
transmitting a composite sensor signal associated with the first and the second parameter values utilizing at least one of the at least one mobile sensor systems, and
decomposing the received composite sensor signal into a first sensor signal representative of the first parameter value and a second sensor signal representative of the second parameter value or decomposing composite.

5. The method according to claim 4, comprising:
converting first sensor data associated with a first sensor signal that is transmitted in a first data format and second sensor data associated with a second sensor signal that is transmitted in a second data format into a first and second converted sensor data, respectively, of identical data format.

6. The method according to claim 5, comprising:
gathering data associated with received sensor signals into a data record.

7. The method according to claim 6, comprising:
making the data associated with the received sensor signal and/or the received back-end system signal available to a user interface of the mobile base unit.

8. The method according to claim 7, wherein the data associated with the received sensor signal and/or the received back-end system signal is communicated to the user interface utilizing Internet Protocol (IP).

9. The method according to claim 6, comprising:
transmitting historical data stored in a memory of the mobile base unit upon request by the back-end system or when triggered by a special event.

10. The method according to claim 9, wherein the at least one sensor system signal is transmitted to the receiver of the mobile base unit by wireless transmission.

11. The method according to claim 10, wherein the transmission to the receiver of the mobile base unit is made through BLUETOOTH® technology.

12. The method according to claim 11, wherein the at least one sensor system is integrated with the mobile base unit.

13. The method according to claim 12, wherein measurements are made continuously or at regular time intervals.

14. The method according to claim 13, wherein the measurements are transmitted to the receiver of the mobile base system continuously or at regular time intervals.

15. The method according to claim 14, wherein the measurements are transmitted to the back-end system continuously or at regular time intervals.

16. The method according to claim 15, wherein a selection of data from the measurement is transmitted.

17. The method according to claim 16, wherein the selection is a selection of a certain time interval of measurement.

18. A method for testing at least one effect of a pharmaceutical substance in a subject to whom the pharmaceutical substance has been administered, wherein the improvement comprises:
utilizing a mobile monitoring system, comprising:
at least one mobile sensor system arranged for detecting a parameter value and transmitting a sensor signal associated with the parameter value; and
a mobile base unit arranged for receiving the sensor signal from the at least one mobile sensor system and for wirelessly transmitting a mobile base unit signal associated with the received sensor signal;
wherein the at least one mobile sensor system and the mobile base unit are arranged to be carried by a movable object or movable organism and
wherein the mobile monitoring system further comprises a back-end system arranged for receiving the mobile base unit signal, thus allowing a wireless communications link between the mobile base unit and the back-end system, and making data associated with the mobile base unit signal available to a user.

19. The method according to claim 18, wherein the mobile base unit is further arranged for wirelessly transmitting a mobile-base-unit-to-sensor-system signal and wherein the at least one mobile sensor system is arranged for receiving the mobile-base-unit-to-sensor-system signal.

20. The method according to claim 19, wherein at least one of the at least one mobile sensor systems is arranged for detecting a first parameter value and a second parameter value and for transmitting a composite sensor signal associated with the first and the second parameter values.

21. The method according to claim 20, wherein the mobile base unit is arranged for gathering data associated with received sensor signals into a data record, and for processing the data record.

22. The method according to claim 21, wherein the mobile base unit is arranged for adding into the data record first sensor data associated with a first sensor signal from a first mobile sensor system received during a predetermined time interval and second sensor data associated with a second sensor signal from a second mobile sensor system received during that predetermined time interval, and subsequently processing the data record.

23. The method according to claim 22, wherein the mobile base unit is arranged to accumulate the received sensor signal and/or sensor data associated with the received sensor signal during a predetermined storage interval, and transmitting the mobile base unit signal associated with the accumulated sensor signal and/or accumulated data associated with the received sensor signal after lapse of the predetermined storage interval.

24. The method according to claim 23, wherein the mobile base unit is arranged for, during consecutive predetermined storage intervals, for each predetermined storage interval accumulating the sensor signals or data associated with the sensor signals received during that predetermined storage interval, and transmitting the mobile base unit signals associated with the accumulated sensor signals or data associated with the received sensor signals after lapse of that predetermined storage interval.

25. The method according to claim 24, wherein the mobile base unit comprises an indicator for indicating data associated with the received sensor signal and/or the received back-end system signal to the user and/or movable organism.

26. The method according to claim 25, wherein the indicator comprises a display, and the data associated with the received sensor signal and/or the received back-end system signal is indicated to the user and/or movable organism via a user interface.

27. The method according to claim 26, wherein the mobile base unit is arranged for making the data associated with the received sensor signal and/or the received back-end system signal available to the user interface.

28. The method according to claim 27, wherein the data associated with the received sensor signal is communicated to the user interface utilizing Internet Protocol (IP).

29. The method according to claim 28, comprising a memory for storing the data associated with the received sensor signal.

30. The method according to claim 29, wherein the at least one mobile sensor system is connected or connectable to the mobile base unit via a wireless communications connection.

31. The method according to claim 30, wherein the mobile base unit is connected or connectable to the back-end system via a wireless communications link.

32. The method according to claim 31, wherein at least one of the mobile sensor systems comprises an event button and is arranged for transmitting a sensor signal associated with a status of the event button.

33. A monitoring system for testing an effect of a pharmaceutical substance in a subject, the monitoring system comprising:
a mobile body area network and a remote user network,
wherein the mobile body area network comprises a sensor system and a mobile base unit and the remote user network comprises a back-end system,
wherein the sensor system is suitable for determining a parameter value of a subject, the parameter value associated with the effect of the pharmaceutical substance, and for transmitting a sensor signal to the mobile base unit, the sensor signal associated with the parameter value, and the mobile base unit is suitable for receiving the sensor signal and for wirelessly transmitting a mobile base unit signal associated with the sensor signal to the back-end system,
wherein the back-end system comprises a service controller, an on-line service center and an interface to a digital vault, the service controller arranged for automatically preparing an exact and secured copy of the at least one mobile base unit signal associated with the received sensor signal and transmitting the exact and secured copy to the digital vault, and the on-line service center arranged for correlating the at least one sensor signal with at least one effect of the pharmaceutical substance.
